(19) United States
(12) Patent Application Publication
(10) Pub. No.: US 2022/0003686 A1
(43) Pub. Date: Jan. 6, 2022

(54) PROBE SUITABLE FOR MEASURING THE COMPOSITION OF AN OXIDISING GAS

(71) Applicant: CMR GROUP, Marseille (FR)

(72) Inventors: Patrice FLOT, Cabries (FR); Mathias DIGNETON, Marseille (FR)

(73) Assignee: CMR GROUP, Marseille (FR)

(21) Appl. No.: 17/294,042

(22) PCT Filed: Nov. 19, 2019
(86) PCT No.: PCT/FR2019/052749

§ 371 Date: May 14, 2021

(30) Foreign Application Priority Data
Nov. 19, 2018 (FR) 1871592

(51) Int. Cl. G01N 21/85; G01N 25/18

(52) U.S. Cl. CPC G01N 21/8507; G01N 2021/3181; G01N 2021/8578; G01N 25/18

(57) ABSTRACT

A probe including a spectrometer adapted to measure the composition of an oxidizing gas after analysis of at least one portion of a light beam having interacted with said gas. The spectrometer includes: a light source adapted to emit a light beam, an optical device comprising optical elements which are configured to guide, towards the gas to be analyzed, at least one portion of the light beam, and guide, towards a detector, a portion of the light beam having interacted with the gas. Further, the probe includes a cage adapted to be removably installed inside a channel through which the gas to be analyzed flows, the cage is configured to permit a flow of the gas through said cage, and at least one portion of the light beam propagates through the cage so as to be able to interact with the gas.

### TECHNICAL FIELD OF THE INVENTION

The object of the invention is a probe adapted to measure the composition of an oxidizing gas. An object of the invention is also a system for measuring the composition of an oxidizing gas including such a probe.

It relates to the technical field of analysis in real time of the composition of an oxidizing gas, and more particularly an analysis via in-situ spectrometry, directly in a channel where the oxidizing gas flows. It applies in particular, but not exclusively, to the monitoring of the quality of the oxidizing gas for the supply of apparatuses that use this gas a fuel.

### PRIOR ART

Measuring the composition of an oxidizing gas is particularly interesting for monitoring the quality of the oxidizing gas for the supply for gas apparatuses of the gas thermal engine, gas burner, gas boiler, gas turbine, gas oven type, etc. The gas can indeed have variations in its composition, more or less rapid, resulting in poor combustion. These variations can in particular come from the source of the gas and/or treatment to which it can be subjected. The changes in the composition of the gas can be sudden when they come from a switching of valves and/or gas supply sources over a network. This is the case when switching from a gas tank to the gas network, or vice-versa. This is also the case when the supply is done via “Boil-Off” of a tank, and when switching from an empty tank to a full tank: in one case it is the last heavy parts of the gas which are sent into the consuming apparatus, while in the case it is the light parts of the gas. According to the control possibilities of the gas apparatus, a sudden change in the quality of the gas can result in an emergency shutdown of the combustion and of the apparatus, for safety reasons.

To optimize the combustion in this type of apparatus, it therefore appears useful to be able to measure in real time the composition of the gas, for example in order to quickly adapt the ejection speed of the gas and/or of the air, and/or the air-gas ratio, and/or ignition advance. Typically, gas engines, with alternative combustion, are sensitive to the methane index and to the calorific value of the oxidizing gas. Gas turbines, boilers, ovens, burners, are sensitive to the Wobbe index, to the calorific value, and to the speed of combustion of the oxidizing gas. These parameters can be determined through calculation from the composition of the oxidizing gas.

Patent documents US2006/0092423 (SERVAITES), U.S. Pat. No. 8,139,222 (SAVELIEV) and U.S. Pat. No. 9,291,610 (ZELEPOUGA) describe probes including a cell wherein a sample of the gas to be analyzed flows. A spectrometer is associated with this cell to measure the composition of the gas. These cells allow a limited flow rate of gas and therefore operate by sampling as derivation of the main gas circuit, which induces a delay in the analysis with respect to the sampling point, a delay that can be of several seconds.

Known more particularly from patent document EP2198277 (SP3H) is a probe that makes it possible to measure the composition of a fluid (including gases). This probe includes a light source emitting a light beam in a conduit or a tank containing the fluid to be analyzed. A spectrometer analyzes the light beam source having interacted with the fluid, to generate measurement data of the composition of said fluid. A set of optical elements makes it possible to guide towards the conduit or the tank, at least one portion of the light beam emitted by the light source, and to guide towards a detector of the spectrometer, the portion of the light beam having interacted with the fluid.

This probe has several disadvantages. In the case where a tank is used, the fluid is static, i.e. it does not flow in said tank. It is therefore understood that it is necessary to fill the tank beforehand with the fluid to be analyzed, before taking the measurement. For a later measurement, the tank has to be emptied before filling it again. This solution is therefore not adapted to a measurement in real time of the composition of the fluid. The steps of filling and emptying the tank prevent any continuous measurement.

In the case where a conduit is used, it is not specified whether the fluid flows in said conduit or on the contrary remains static. It nevertheless remains that an optical detector, or an optical reflector has to be permanently installed in the conduit and permanently attached to an internal wall of the latter. It therefore appears necessary to modify the distribution network of the fluid to be analyzed in order to permanently connect this specific conduit thereto. This installation can be complex and expensive. The same applies when an intervention has to be carried out on the probe, and more particularly on the optical portion, detector or reflector, facing the light source, and when it is necessary to isolate the distribution network from the fluid in order to disassemble the specific conduit. The same problems appear when the reflector is permanently attached in the tank.

Known in U.S. Pat. No. 4,549,080 (LOWELL) is a probe that includes a cage adapted to be removably installed inside a channel wherein the gas to be analyzed flows. The cage is configured to permit a flow of the gas through said cage. At least one portion of the light beam propagates through the cage so as to be able to interact with the gas. The cage serves as a holder for an optical reflector adapted to reflect, towards the detector, the portion of the light beam propagating through said cage and having interacted with the gas. The cage consists of a porous ceramic filter in such a way that the gas flows with difficulty through said filter. Indeed, the gas undergoes a strong constraint and has to handle great resistance to pass through the ceramic wall. This results in that the measurements taken by the probe are not in real time, or at the very least with a certain delay, due to the latency of the gas to pass through the ceramic wall.

The invention aims to overcome this situation. In particular, an objective of the invention is to propose a probe that makes it possible to measure in real time and/or with an optimized response time, the composition of an oxidizing gas flowing in an existing channel and that is not specifically designed for this purpose.

Another objective of the invention is to propose a measurement probe that can quickly and easily installed/removed from a channel.

Yet another objective of the invention is to propose a measurement of which the design is simple, robust and of which the costs of manufacturing are particularly reduced.

### DISCLOSURE OF THE INVENTION

The solution proposed by the invention is a probe comprising a spectrometer adapted to measure the composition of an oxidizing gas after analysis of at least one portion of a light beam having interacted with said gas, said spectrometer including:
a light source adapted to emit a light beam,
an optical device including optical elements configured:
   to guide, towards the gas to be analyzed, at least one portion of the light beam, and
   to guide, towards a detector, a light beam portion having interacted with the gas.

And wherein:
the probe includes a cage adapted to be removably installed inside a channel wherein the gas to be analyzed flows,
the cage is configured to permit a flow of the gas through said cage,
at least one light beam portion propagates through the cage so as to be able to interact with the gas,
the cage serves as a holder for an optical reflector adapted to reflect, towards the detector, the light beam portion propagating through said cage and having interacted with the gas.

This probe is remarkable in that the cage is formed by an elongate tube the sidewall of which contains apertures distributed over the perimeter of said wall, said apertures are configured to allow the gas to pass radially through said tube, the combined area of said apertures more than 50% of the area of the wall of said tube, and preferably more than 80%.

Intended for industrial applications, this probe is designed to resist harsh environments: polluted atmosphere, high ambient temperature, environment passed through by radio waves, vibrations. In particular, its design does not include any moving parts.

This probe can be removably installed on an existing channel Generally, the supply channels of gas apparatuses, are equipped with flanges, or other similar arrangements, that allow for the installation of temperature or pressure probes for example. The probe object of the invention can indeed be mounted in this way using a single flange, without it being necessary to modify the channel.

No intervention must be carried out on the channel itself in that the latter does not integrate any optical element required for the measurement. Indeed, the optical reflector is now borne by the removable cage, not by the channel.

Furthermore, through the configuration of the apertures of the cage, the gas flows freely and continuously inside the cage (without any additional means of pumping), with the least constraints possible, in such a way that the probe allows for a measurement in real time of the composition of the gas, with an optimized response time.

Other advantageous characteristics of the probe object of the invention are listed hereinbelow. Each one of these characteristics can be considered individually or in combination with the remarkable characteristics defined hereinabove, and be the object, where applicable, of one or more divisional patent applications:
Advantageously, the optical device includes an optical element adapted to guide, towards a second detector of the spectrometer, a portion of the light beam emitted by the light source in such a way that said spectrometer also analyses a light beam portion that does not propagate through the cage; the spectrometer is adapted to measure the composition of the gas according to the data resulting from the analysis of the light beam portion that has propagated through the cage, and the data resulting from the analysis of the light beam portion that does not propagate through the cage.
According to an embodiment, the optical element is an optical separator placed between the light source and the cage, said optical separator is adapted to:—transmit in the cage, a portion of the light beam emitted by the light source; —deviate towards a first detector, the light beam portion that has propagated through the cage and which is reflected by the optical reflector arranged in said cage; —deviate towards the second detector of the spectrometer, the light beam portion that does not propagate through the cage.
According to another embodiment, the optical device includes an optical element adapted to guide:—towards the cage, a portion of the light beam emitted by the light source; —towards the detector, the light beam portion having propagated through the cage and which is reflected by the optical reflector arranged in said cage.
Advantageously, an air gap serves as an interface between the light source and the optical separator; and an air gap serves as an interface between the optical separator and the cage.
An air gap also advantageously serves as the interface between the optical separator and the first detector. A first lens and a first diaphragm are disposed in the air gap that separates the optical separator and the first detector, said first lens and said first diaphragm being arranged in such a way that said beam portion having propagated through the cage and which is reflected by the optical reflector, passes firstly through said first lens then said first diaphragm before impacting said first detector.
An air gap also advantageously serves as the interface between the optical separator and the second detector. A second lens and a second diaphragm are disposed in the air gap that separates the optical separator and the second detector, said second lens and said second diaphragm being arranged in such a way that the light beam portion that does not propagate through the cage, first passes through said second lens then said second diaphragm before impacting said second detector.
According to an embodiment, a thermal conductivity sensor is arranged on the probe in such a way that said sensor can interact with the gas to be analyzed; a data processing unit is adapted to generate measurement data of the composition of the gas by taking account of the signals emitted by the thermal conductivity sensor and the measurement data generated by the spectrometer.
Advantageously, the probe includes a gas-tight chamber to be analyzed and wherein the spectrometer is installed. The cage includes a first end and a second end that are opposite, said first end includes an aperture that communicates with the chamber. The optical reflector, the aperture and the light source are aligned along the same axis.
A transparent window made from polished sapphire is preferably disposed facing the aperture arranged at the first end of the cage, said window forms the gas seal between the chamber and said cage, the portion of the light beam propagating through said cage, passant through said window.
According to an embodiment, the optical reflector is installed on an endpiece, said endpiece is added onto an end of the cage.
Advantageously, a transparent window made of polished sapphire covers the optical reflector.
According to an embodiment, the probe includes a gas-tight chamber to be analyzed and wherein the spectrometer is installed, with more than 50% of the volume of said chamber being filled with resin or elastomer.

Another aspect of the invention relates to a system including a probe, said probe comprises a spectrometer adapted to measure the composition of an oxidizing gas after analysis of at least one portion of a light beam having interacted with said gas, said spectrometer including:
a light source adapted to emit a light beam,
an optical device including optical elements configured:
   to guide, towards the gas to be analyzed, at least one portion of the light beam, and
   to guide, towards a detector, a light beam portion having interacted with the gas.

And wherein:
the system includes a channel wherein the gas to be analyzed flows,
the probe is in accordance with one of the preceding characteristics, the cage of said probe being removably installed inside the channel, said cage is configured in such a way that the gas flows through said cage,
at least one portion of the light beam propagates through the cage and interacts with the gas,
the cage serves as a holder for an optical reflector adapted to reflect, towards the detector, the portion of the light beam propagating through said cage and having interacted with the gas.

Other advantageous characteristics of the system object of the invention are listed hereinbelow. Each one of these characteristics can be considered individually or in combination with the remarkable characteristics defined hereinabove, and be the object, where applicable, of one or more divisional patent applications:
According to an embodiment, the cage has a longitudinal axis perpendicular or substantially perpendicular to the direction of flow of the gas in the channel.
When the channel has an elbow, the cage has a longitudinal axis parallel or substantially parallel to the direction of flow of the gas in the elbow of the channel
According to an embodiment, the system further includes a gas chromatography apparatus adapted to generate measurement data of the composition of the gas, said apparatus is connected to the channel, in such a way that a sample of the gas flowing in said channel is analyzed by said apparatus.
The probe and the gas chromatography apparatus are advantageously connected to a data processing unit; said processing unit is adapted to correct the measurement data generated by the probe according to the measurement data generated by the gas chromatography apparatus.
According to an embodiment, the probe further includes a thermal conductivity sensor arranged in such a way that said sensor can interact with the gas to be analyzed. A data processing unit is adapted to generate measurement data of the composition of the gas by taking account of the signals emitted by the thermal conductivity sensor and measurement data generated by the spectrometer. The probe and the gas chromatography apparatus are connected to a data processing unit, said processing unit is adapted to correct said measurement data of the composition of the gas according to said measurement data generated by the gas chromatography apparatus.

### DESCRIPTION OF THE FIGURES

Other advantages and characteristics of the invention shall appear better when reading the following description of a preferred embodiment, in reference to the accompanying drawings, which are non-limiting and given as examples and wherein:

FIG. 1A is a perspective view of a probe in accordance with the invention without fastening flange,

FIG. 1B is another perspective view of a probe in accordance with the invention without fastening flange,

FIG. 1C shows the probe of FIG. 1A with fastening flange,

FIG. 2 is a partial cross-section view of a probe in accordance with the invention,

FIG. 3 diagrams an embodiment of a probe in accordance with the invention,

FIG. 4 diagrams another embodiment of a probe in accordance with the invention,

FIG. 5 further diagrams another embodiment of a probe in accordance with the invention,

FIG. 6A diagrams the installation of a probe in accordance with the invention on a straight section of the channel, following a mounting perpendicular to the axis of the channel,

FIG. 6B diagrams the installation of a probe in accordance with the invention on an elbowed section of the channel, according to a coaxial mounting to a branch of the channel,

FIG. 6C diagrams the installation of a probe in accordance with the invention on an elbowed section of the channel, according to another coaxial mounting to a branch of the channel,

FIG. 6D diagrams the installation of a probe in accordance with the invention on a straight section of the channel, following another mounting perpendicular to the axis of the channel,

FIG. 7 diagrams the installation, on a channel, of a probe in accordance with the invention and of a chromatography apparatus,

FIG. 8 is a graph showing a continuous measurement of the concentration of a gas via spectrometry, one-off measurements of the concentration of the gas by chromatography, and the result of the continuous measurement by spectrometry corrected by the one-off measurements by chromatography,

FIG. 9 diagrams a probe in accordance with the invention, in an alternative embodiment.

### PREFERRED EMBODIMENTS OF THE INVENTION

The probe object of the invention is used to measure the composition of an oxidizing gas. The latter is more particularly a gas used as a fuel in an apparatus of the type gas thermal engine, gas burner, gas boiler, gas turbine, gas oven, etc. The gas can also flow in a channel that can be part of a distribution network of the gas pipeline type.

The probe is in particular used to control apparatuses that burn gas, regardless of the variability in the quality of the gas. It allows for better control of the apparatus when the quality of the gas can change, by acting on one or more parameters such as: ejection speed of the gas and/or of the air, air-gas ratio, ignition advance, etc.

The gas to be analyzed can for example be methane (CH₄), ethane (C₂H₆), propane (C₃H₈), iso-butane (i-C₄H₁₀), normal-butane (n-C₄H₁₀), hydrogen (H₂), carbon monoxide (CO), carbon dioxide (CO₂), or a gaseous mixture of these compounds.

The measurement of the composition of the gas is to be understood as the determination of the concentration (%), by weight or by volume, of one or more compounds of said gas, in proportions that are able to affect combustion.

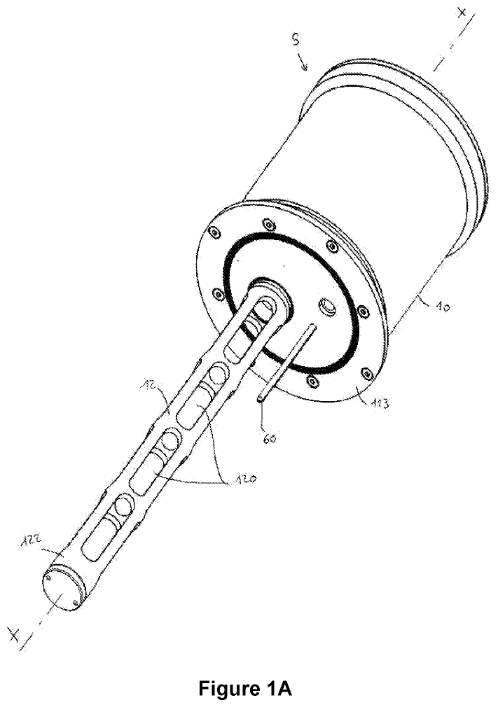

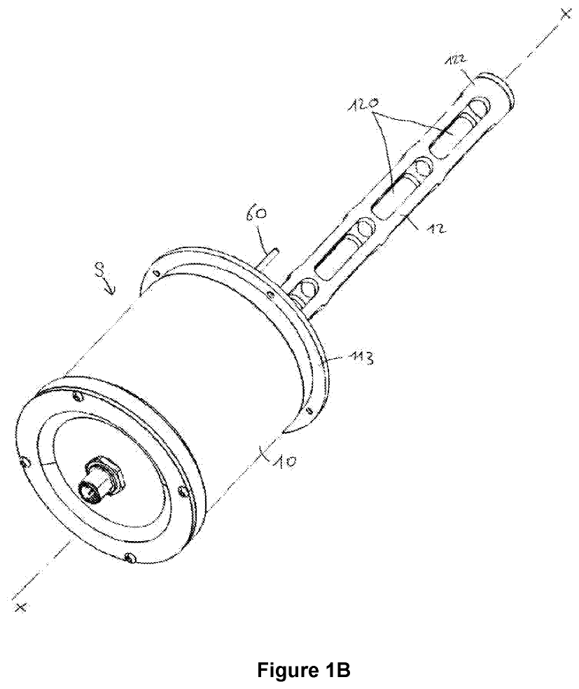

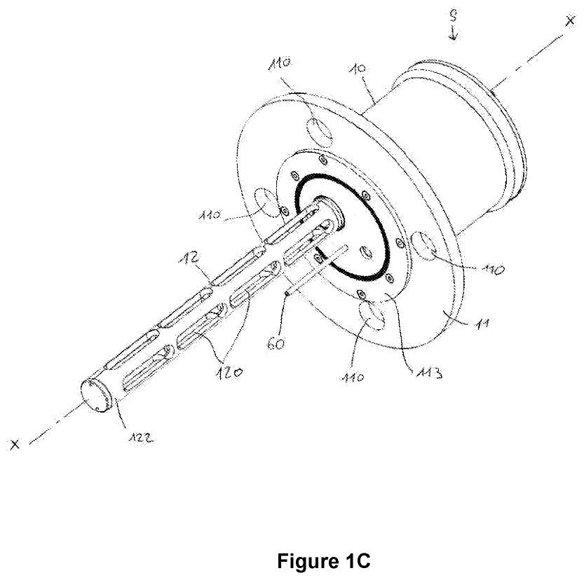

In FIGS. 1A 1B and 1C, the probe S includes a cover 10 delimiting a gas-tight chamber to be analyzed. For the purposes of illustration, the cover 10 has a general cylindrical aspect of which the diameter is comprised between 5 cm and 20 cm and the height comprised between 5 cm and 30 cm. The capot 10 is advantageously made from stainless steel, to arrange a Faraday cage effect (for the case where the probe S is used in an environment passed through by radio waves) as well as anti-corrosion protection, and can be obtained by molding or machining. In the rest of the description, the terms “capot” and “chamber” will be used in a similar manner.

In FIG. 1C, the chamber 10 is engaged with a fastening flange 11 allowing for the fixation of the probe S on a channel wherein the gas to be analyzed flows. This flange 11 has the shape of a flat disk fastened to a base of the chamber 10 and of which the dimensions are greater than those of said chamber. This base can be seen in particular in FIGS. 1A and 1C and bears reference 113. The base 113 facilitates the mounting of the flange 11, but is however optional. The flange 11 for example has a diameter comprised between 10 cm and 40 cm and a thickness comprised between 1 cm and 5 cm. The flange 11 has drillings 110 wherein threaded bolts or rods are engaged for the engagement to the channel, preferably according to standard NF EN 1092 for flat flange in PN 10. The flange 11 is advantageously made from stainless steel and can be obtained by molding, stamping or machining. The fastening of the flange 11 onto the base 113 of the chamber 10 can be carried out by welding, screwing, bolting, riveting, etc. The flange 11 can also be mounted mobile in rotation around the base 113, along the axis of the chamber 10. In this case, the engagement of the flange 11 on the base 113 can for example be carried out by clamping, using threaded bolts and/or rods.

A cage 12 extends opposite the chamber 10, along a longitudinal axis X-X parallel or coaxial to that of said chamber.

The cage 12 can be arranged in such a way that its axis X-X is off-axis in relation to that of the chamber 10. Such an off-axis- or off-center-configuration makes it possible to optimize the positioning of the elements inside the chamber 10, and in particular the elements of the optical device and those for the measurement of pressure and temperature. The dimensions of the chamber 10 can then be reduced with respect to a configuration where the axis X-X of the cage 12 is coaxial to the axis of the chamber 10.

In FIGS. 1A and 1B, the cage 12 is formed by an elongate tube of circular section, in such a way that said tube has a general cylindrical shape. This tube 12 for example has a length comprised between 3 cm and 50 cm and an outer diameter comprised between 1 cm and 10 cm. The thickness of the sidewall of the tube 12 is for example comprised between 0.1 cm and 1 cm. The tube 12 is advantageously made from stainless steel and can be obtained by molding or machining. The tube 12 is engaged with the base 113 of the chamber 10, the fastening able to be carried out by welding, screwing, bolting, riveting, etc.

The probe S is therefore shaped by: the chamber 10 (and optionally its base 113), the flange 11 and the cage 12. In order to optimize the compactness of the probe S, the elements 10, 113 and 11 are coaxial.

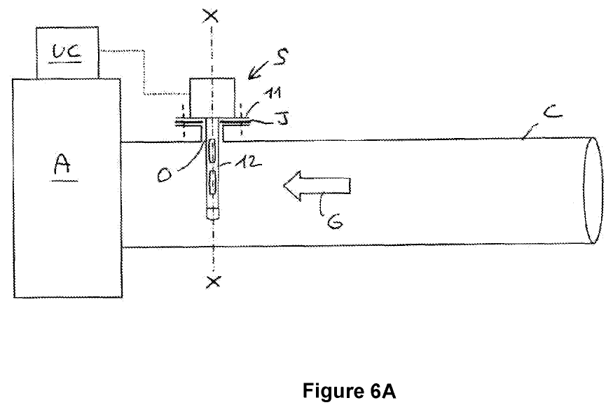

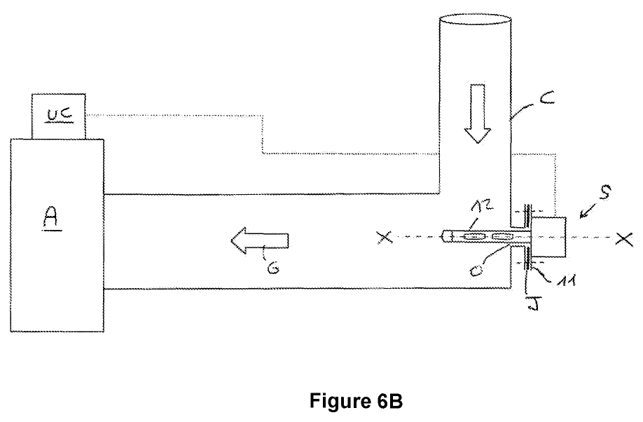

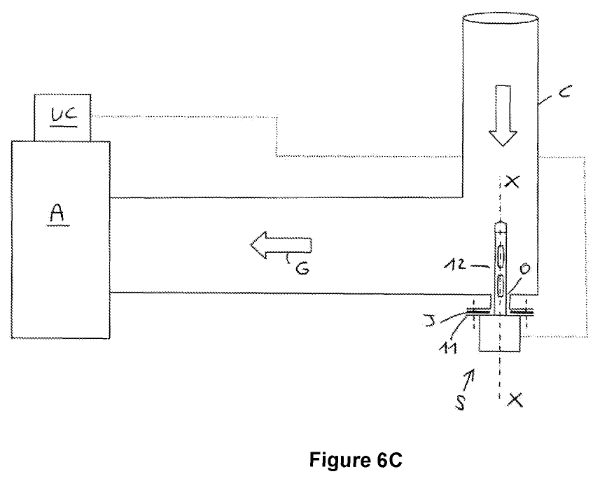

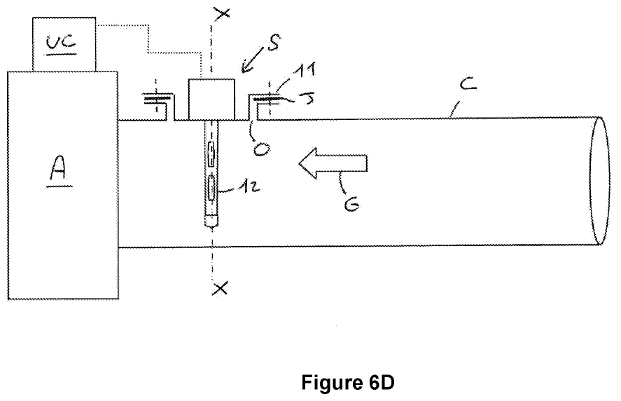

In reference to figures IA, 1B, 1C and 2, the sidewall of the tube 12 contains apertures 120 that are distributed over the perimeter of said wall. These apertures 120 allow the gas to be analyzed to freely and quickly flow inside the cage 12 when the latter is plunged into a channel where said gas flows. These apertures 120 form meshes that allow the gas to penetrate into and to exit the tube 12. In the assembly of FIGS. 6A and 6D, the gas passes through the cage 12 along in particular a radial component. In other terms, the gas penetrates and exits radially the tube 12 via the apertures 120.

According to a preferred embodiment that allows the gas to pass through the tube 12 with the least constraints possible, the apertures 120 are arranged over most of the sidewall of said tube. Advantageously, the combined area of the apertures 120 represents more than 50% of the area of the wall of the tube 12, and preferably more than 80%. The shape of the apertures 120 can be chosen in order to create possible turbulences inside the tube 12 able to homogenize the gaseous mixture in the tube 12 and improve the response time of the probe S without disturbing the measurement. In order to facilitate the flow of the gas through the tube 12, the apertures 120 are advantageously disposed in such a way as to be radially opposite. Correct operation of the probe S is however obtained when the apertures 120 are not radially opposite. The apertures 120 can be of an oblong, circular, square, rectangular, or other shape, and by obtained by machining of the tube 120. Their number can for example vary from 2 to 100. The shape and/or the disposition of these apertures 120 can be defined and optimized by a calculation of the aerodynamic flows inside and around the cage 12, using a software from the market such as Ansys Fluent®.

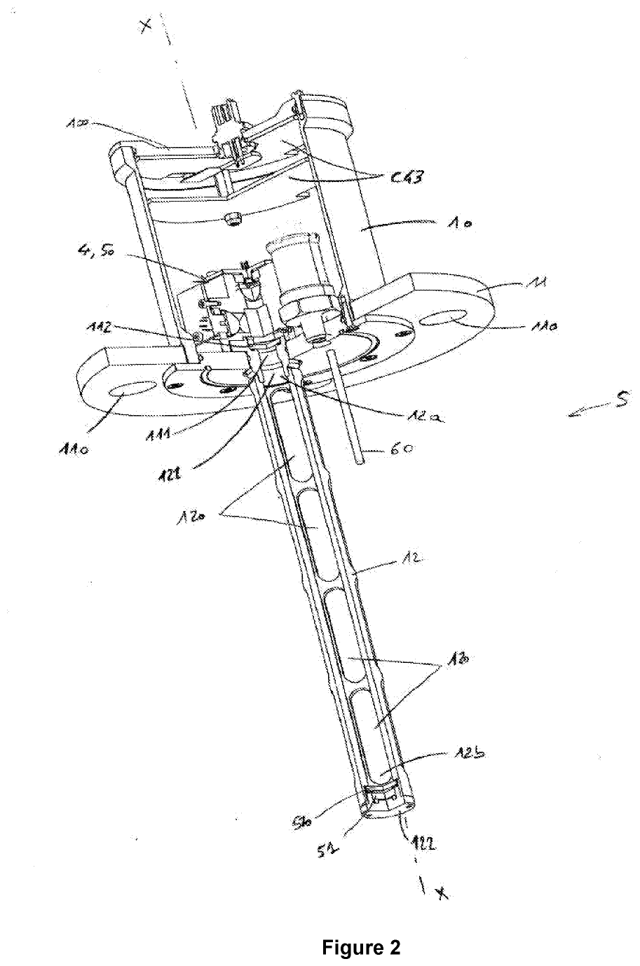

In reference to FIG. 2, the cage 12 includes a first end 12a and a second end 12b that are opposite. The first end 12a has an aperture 121 that communicates with the chamber 10. More particularly, when the cage 12 is fastened on the base 113 of the chamber 10, the aperture 121 is positioned in a passage 111 made in said base, said passage opens into the chamber 10. The aperture 121 and the passage 111 have a section (shape and dimension) that correspond to that of the cage 12.

A transparent window 112 is disposed facing the aperture 121. In FIG. 2, this window 112 is inserted into the passage 111. It forms the gas seal between the chamber 10 and the cage 12, in such a way that the gas flowing through said cage cannot penetrate inside said chamber. The chambre 10 is therefore sealed to the gas to be analyzed. As an example, the dimensions of this window are comprised between 10 mm and 20 mm in diameter and between 1 mm and 5 mm in thickness. This range of thickness makes it possible to resist the pressure of the gas flowing through the cage 12, without deformation of the window 112, and therefore without impact on the light beam.

The window 112 is preferably made of polished sapphire. This material has the main advantage of not attracting dust, or the condensates contained in the gas to be analyzed, thus preventing the fowling and the darkening over time of said window even if precautions are taken to close the chamber 10 in a dry and clean atmosphere during manufacture, and in a sealed manner. Furthermore, sapphire has the property of being practically transparent to infrared light in such a way that the passing of the portion of the light beam through the window 112 results in little loss of light power (little absorption and little reflection). Using this material does not require anti-reflective treatment on the window 112.

An endpiece 122 is added onto the second end 12b. This endpiece 122 has for example the shape of a cylindrical which closes off the second end 12b. This endpiece 122 is advantageously made from stainless steel and can be obtained by molding or machining. It is engaged with the cage 12 by welding, screwing, bolting, riveting, etc. The endpiece 122 has for example a length comprised between 1 cm and 3 cm and an inner diameter substantially corresponding to the outer diameter of the cage 12. The thickness of the sidewall thereof is for example comprised between 0.1 cm and 1 cm.

The endpiece 122 serves as a housing for an optical reflector 51. As explained hereinabove in the description, this reflector 51 is adapted to reflect, towards a detector of a spectrometer 4, a portion of a light beam propagating through the cage 12. In order to optimize the optical path, the reflector 51, the aperture 121, the passage 111 and the light source 40 are aligned along the same axis X-X.

According to a preferred embodiment that makes it possible to reconcile robustness, precision and low cost, the reflector 51 is a concave spherical mirror made from a silicon support whereon is located, exposed to the light beam, a polished aluminum deposit and covered with a surface hardening treatment. This mirror 51 allows not only to concentrate the light beam, but also to carry out a desired enlargement voulu of this beam.

The radius of the sphere of the mirror 51 is for example comprised between 100 mm and 250 mm. As explained hereinabove in the description, the radius of curvature depends on the length of the optical path between the light source 40 and a detector 41 of the spectrometer 4 (FIG. 2). The shorter the optical path is, the smaller the radius of curvature will be. The oxidizing gas to be analyzed generally flows under pressure in the channel (generally a relative pressure from 0 to 10 bars, but which can be higher). Up to an absolute pressure P of 60 bars, the length L in cm of the optical path to be considered, that passes through the gas between the window 112 and the reflector 51, approximately follows the following formula: L=200/P. A high pressure of the gas can therefore require reducing the length of the optical path and, because of this, reduce the radius of curvature of the mirror 51. From the application of this formula, the length L can be optimized experimentally to maximize the signal/noise ratio of the absorption over the entire operating range in pressure, and temperature of the probe S.

The radius of curvature of the mirror 51 also depends on an optional enlarging to be done between the actual object (the light source 40) and the image (the sensitive surface of the detector 41 of the spectrometer 4).

The reflector 51 is covered by a transparent window 510. This window 510 has the function of isolating the reflector 51 from the gas to be analyzed, and forms for this purpose a gas seal. The material of the window 510 is advantageously polished sapphire, for the same reasons as those mentioned hereinabove.

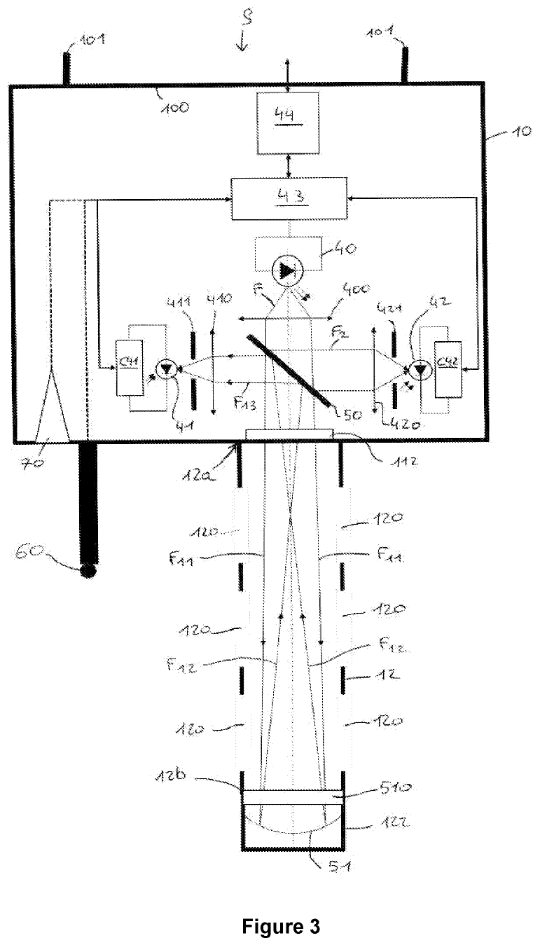

In the embodiment of FIG. 3, the chamber 10 houses in particular all the electronic components of the probe S, optical elements of the spectrometer 4 and the light source 40 adapted to emit a light beam F.

The spectrometer 4 is adapted to generate measurement data of the composition of a gas after analysis of at least one portion of the light beam F having interacted with said gas. This analysis is based on absorption spectroscopy, and more particularly on the near-infrared absorption spectroscopy. This method well known to those skilled in the art makes it possible to determine the concentration of a gaseous compound by measuring the intensity of the electromagnetic radiation that it absorbs at different wavelengths. The spectrometer 4 includes for this purpose two detectors 41 and 42 which are standard and known to those skilled in the art. They are each formed from one or more photosensitive cells (CCD, CMOS, . . . ) adapted to transform the captured photons into electric signals. These detectors 41, 42 can each be disposed on a printed circuit board C41, C42.

The best results in terms of measurement precision are obtained when the light source 40 emits in the near infrared, over a wavelength spectrum comprised between 1550 nm and 1850 nm. In order to reduce costs, the light source 40 preferably consist in an assembly of one or more LEDs (light-emitting diodes). Four LEDs are advantageously used to cover the entire spectrum and to have enough light powered.

For reasons of compactness and simplification of the mounting, the various LEDs are advantageously integrated into the same electronic component. In addition, LEDs have the advantage of having a low thermal emissivity. The applicant has been able to observe in the prototyping phase that the temperature of the LEDs with respect to the ambient temperature is increased by about 6° C., thereby reducing the risks of explosion in case of a gas leak in the chamber 10. In addition to its low cost, the advantage of LEDs with respect to a lighting means of the laser type, is a lower geometrical requirement, as laser is much more directive. This results in a decrease in the cost of parts and labor and fast mounting.

The LEDs are powered by a current pulsed at a certain frequency and regulated on a fixed setpoint of current intensity: there is no active or dynamic adjustment of the intensity of the control current of LEDs. Advantageously, the intensity of the current is adjusted on a very high setpoint, for example 200 mA, even 2 A by LED according to their type, so as to obtain maximum light power.

The power supply current of the light source 40 can come from a source of current integrated into the chamber 10 in the form of a battery, or come from an external source of current. In this case, the probe S includes a power supply cable that allows it to be plugged into this external source of current.

An optical element 50 placed in the chamber 10, makes it possible to guide through the cage 12, a portion F₁₁ of the light beam F. This optical element is advantageously an optical separator placed between the light source 40 and the cage 12. The optical separator 50 has a cubic shape (separator cube). It is formed from two prisms assembled to one another on their largest face (the hypotenuse). These larger faces are inclined 45° with respect to the incident beam F. The assembly can be done via gluing. The side of this cube has a length that corresponds to the diameter of the window 510. This length can however be slightly smaller when it is greater than or equal to the width of the light beam F that it impacts. The separator cube 50 preferably has an anti-reflective treatment. In order to optimize the optical path, the optical separator 50 is placed on the axis X-X, in the same alignment as the reflector 51, the aperture 121, the passage 111 and the light source 40.

The air gap serves as an interface between the light source 40 and the optical separator 50. Contrary to the aforementioned patent document EP2198277, no light guide, or optical fiber are used, which makes it possible to reduce the costs and simplify the design of the probe S.

In general, the light source 40 emits light rays in all directions. So, a collimation lens 400 is disposed between the light source 40 and the optical separator 50. This lens 400 makes it possible to converge the rays of the beam F towards the separator 50. By placing the light source 40 very close in front of the focal point of this lens 400, the rays emerging from said lens are quasi parallel and slightly converging. The lens 400 is a standard collimation lens that, advantageously, has no undergone any anti-reflective treatment with the goal of reducing costs.

In FIG. 3, the separator 50 laisse passer a portion F₁₁ of the light beam F towards the cage 12 and deviates another portion F₂ according to an angle of 90°. The separator 50 therefore carries out a first angle deflection at 90° before the propagation of the light flow through the cage 12. The portion F₂ therefore does not interact with the gas to be analyzed. This portion F₂ impacts the second detector 42 of the spectrometer 4.

The portion F₁₁ passes through the separator 50 in the same direction as the beam F, without deviation. This portion F₁₁ passes through the window 112 and penetrates into the cage 12 in order to interact with the gas G to be analyzed. It is also observed here than an air gap serves as an interface between the separator 50 and the cage 12, as no optical fiber is used. The portion F₁₁ then passes through the window 510 to be reflected by the reflector 51. The reflected portion F₁₂ follows the reverse path of the portion F₁₁ and interacts again with the gas G. There is therefore a double interaction of the light flow with the gas G in the cage 12: “an outward” interaction on the portion F₁₁ and a “return” interaction on the portion F₁₂. The reflected portion F₁₂ passes through the window 112 and penetrates into the chamber 10 to impact the separator 50. The separator 50 deviates the portion F₁₂ according to an angle of 90° (portion F₁₃). The separator 50 therefore carries out a second angle deflection at 90° after the propagation of the light flow through the cage 12, i.e. after the interaction with the gas to be analyzed. The portion F₁₃ therefore impacts the first detector 41 of the spectrometer 4.

The spectrometer 4 can thus measure the composition of the gas G according to:
data resulting from the analysis of the portion F₁₃ of the light beam having propagated through the cage 12 (and having impacted the first detector 41), and
data resulting from the analysis of the portion F₂ of the light beam that does not propagate through the cage 12 (and having impacted the second detector 42).

This measurement is taken in less than one second, in such a way that the probe S is capable of measuring the instantaneous variations of the composition of the gas and of allowing for a very precise control of gas apparatuses in order to provide optimum combustion.

The signals emitted by the detectors 41 and 42 are transmitted to a processing unit 43 of the spectrometer, said unit is adapted to process these signals and generate measurement data of the composition of the gas G. The measurement method is well known to those skilled in the art and is not part of the invention.

The processing unit 43 has the form of one or more printed circuit boards carrying the electronic components that make it possible to generate the measurement data and the electrical power supply of the light source 40. The processing unit 43 in particular comprises one or more processors (including microprocessors and/or FPGA (Field-Programmable Gate Array) and/or microcontrollers), and one or more memories. One or more computer applications—or computer programs—are recorded in the memory or memories and of which the instructions (or code), when they are executed by the processor or processors make it possible to perform the functionalities of the spectrometer 4 and more generally the functionalities of the probe S. In this latter case, the processing unit 43 is common to several functional elements of the probe S, of which for example: a temperature sensor of the gas, a temperature sensor of the LEDs, temperature sensors of each detector 41, 42, a pressure sensor of the gas, and a thermal conductivity sensor of the gas described hereinabove in the description. The processing unit 43 can be associated with a wired communication module 44 (Ethernet or CAN protocol type) or wireless module (Wi-Fi or Bluetooth emitter/receiver type) adapted to receive instructions and/or emit results of the measurements taken in particular digitally.

The acquisition cycles of the measurements and calculations are done by an alternation of turning the light source 40 on and off. The light beam F is emitted by intermittence, preferably at the frequency of 16 kHz (+/−1 kHz), and synchronized with the reception of the light flow on the detectors 41, 42. More precisely at each acquisition and calculation cycle, which can last for example 500 ms, the beam F is activated by successive sequences of a few milliseconds so as to illuminate the detectors 41, 42 successively on their various acquisition frequencies. At the end of this frequency scanning, the beam F is turned off during the time of the calculation cycle, or over a significant period of this cycle, such as for example 100 ms. These various phases of extinction, even of very short duration, caused by the pulse mode, the frequency scanning and the calculation time, total up to for example 40% of the time and make it possible to cool the light source 40 which heats up as soon as it is turned on.

According to a preferred embodiment that makes it possible to reduce the costs of manufacturing, there is no temperature adjustment in the chamber 10. A particular arrangement of the electronic components in the chamber 10 make it possible to reduce the temperature effects on the detectors 41, 42. In particular, the motherboard carrying the processing unit 43 and the electronic components making it possible to manage the electrical power supply of the light source 40, is geographically far from the light source 40 and the boards C41, C42 carrying the detectors 41, 42. This motherboard is the main source of heat. The geographical moving away is for example comprised between 20 mm and 250 mm. In order to have maximum separation, the motherboard is installed on the rear of the chamber 10, opposite the optical portion which is close to the gas to be analyzed. In FIG. 2, the motherboard C43 is thrust on the internal surface of the bottom wall 100 of the chamber 10, said wall is opposite the cage 12. The bottom wall 100 is preferably made of metal, so as to facilitate the exchange of heat with the exterior of the chamber 10. In FIG. 3, cooling fins 101 are arranged on the external surface of the bottom wall 100, said fins accentuate the transfer of calories towards the exterior of the chamber 10. According to the quality of the electronic components retained, these fins are however not necessary.

The use of LEDs, the pulse emission of the light beam F and the particular arrangement of the motherboard, are combined to limit the heating in the chamber 10. In the prototyping phase, the applicant observed a rise in the temperature of the detectors of only 3° C. with respect to the ambient temperature. So, the probe S is perfectly adapted to operate in ATEX (Explosive Atmosphere) zones.

For industrial use, it appears important that the probe S can operate in its environment without producing any electromagnetic disturbances that are detrimental for everything that is inside the Faraday cage that the chamber 10 forms, (electromagnetic compatibility EMC), or suffer from them if such electromagnetic disturbances are produced. To do this, shielded connection cables are used inside the chamber 10, to connect the various components and/or boards. The cables used are for example formed from bundles of wires, for example 10 wires, which have an alternation of working wires and wires connected to the ground. Each ground wire carries out the shielding for two working wires that are adjacent to it.

To secure the use of the probe S even further in the case of an ATEX zone, most of the chamber 10 can be filled with a resin or with an elastomer (for example a “compound”), that has conventional properties of good thermal conductivity and electrical insulation, as commonly used in electronic cases. The volume of air inside the chamber 10 is thus minimized, which makes it possible to limit, even annihilate, the risks of explosion in case of a gas leak in the chamber 10 and/or the harmful effects of condensation and impurities in the air. Advantageously more than 50% of the volume of the chamber 10 is filled with resin or elastomer, and preferably 90% of said volume. The optical path of the spectrometer 4 (light source 40, separator 50 and detectors 41, 42) is left free, i.e. the spaces between the components are not filled with resin or elastomer.

As explained hereinabove, there is no regulation of the control current of the light source 40. Not controlling this current, and optionally, the absence of control of other parameters (temperature, aging), make for a light intensity of the beam F that is variable over time. The two detectors 41 and 42 make it possible to overcome this problem by controlling this variation in light intensity. The first detector 41 measures the intensity of the beam portion F₁₃ (measurement intensity) after passing through the gas to be analyzed. And the second detector 42 measures the intensity of the portion F₂ of the light beam (reference intensity) before the passing in the gas G to be analyzed. Applying Beer-Lambert's law to the two signals gives the light intensity absorbed by the gas. The absorbance A of the gas satisfies the law:

A=log(Io/I)

where Io is the signal measured by the second detector 42 obtained via extrapolation of the reference signal, and I is the signal measured by the first detector 41.

The deviations linked to the temperature of the components with respect to the ambient temperature, and potential constraints in the variation of the current, aging of the LEDs, etc., are thus overcome. The analysis of the portion F₂ of the light beam makes it possible to maintain the calibration of the spectrometer 4. For the measurements, the processing unit 43 takes account of any variation in the light intensity and/or in the frequency spectrum of the light beam F.

To provide corrections to the processing of the signal, the probe S can include a temperature sensor of the LEDs and temperature sensors of each detector 41, 42, with these sensors being integrated into the chamber 10. For the same reasons, a pressure sensor in contact with the gas and/or a temperature sensor (reference 60 in FIGS. 1A, 1B and 1C), in contact with the gas, can be arranged on the base 113. The corrections made by these sensors are known in the prior art and for example mentioned in the aforementioned patent document U.S. Pat. No. 9,291,610 (ZELEPOUGA). The temperature sensor of the gas 60 advantageously has the form of a rod that protrudes from the base 113. A platinum resistance probe (or another similar sensor) is housed in this rod, at the end that is the farthest from the chamber 10. This arrangement makes it possible to maintain the temperature sensor 60 at a distance from the chamber 10. As the latter does not have the same temperature as the gas, it does not influence the measurement.

An air gap (not an optical fiber) provides the interface between the separator 50 and the first detector 41. A first lens 410 and a first diaphragm 411 are disposed in this air gap. The beam portion F₁₃ first passes through the first lens 410 then the first diaphragm 411 before impacting the first detector 41. The first lens 410, the first diaphragm 411 and the first detector 41 are aligned according to the same optical axis. Preferably, the first diaphragm 411 is thrust on the first lens 410. It is preferable to not install an optical filter in front of the first detector 41 in order to retain optimum light power. Indeed, an optical filter is able to reduce the light power by about 10%.

The first lens 410 is a standard converging lens. In order to reduce costs, the first lens 410 does not undergo any anti-reflective treatment. It makes it possible to converge the rays of the beam portion F₁₃ towards the photosensitive face of the first detector 41, in such a way as to optimize the capturing of the photons at said first detector.

Despite the first lens 410, the beam portion F₁₃ that impacts the first detector 41, can have a cone angle that is excessive with respect to the photosensitive face of said detector. Parasite reflections are then produced in detector 41 which disturb the spectral resolution of said detector, thus resulting in a degradation of the quality of its response. The first diaphragm 411 makes it possible to solve this problem for this first detector 41 by using only one lens. It limits the angle of the cone of the beam portion F₁₃ and reduces the parasite reflections IN the first detector 41. The aperture thereof is calculated to respect the maximum cone angle specified for this type of detector, from the distance between the diaphragm 411 and the detector 41. Software in the market such as OpticStudio® developed by Zemax make it possible for example to perform these calculations. The diameter of the aperture of the first diaphragm 411 is for example comprised between 100 μm and 4000 μm.

A similar mounting is provided at the second detector 42. An air gap (not an optical fiber) serves as an interface between the separator 50 and the second detector 42. A second lens 420 and a second diaphragm 421 are disposed in this air gap. As the beam portion F₂ has not interacted with the gas G, first passes through the second lens 420 then the second diaphragm 421 before impacting the second detector 42. The second lens 420, the second diaphragm 421 and the second detector 42 are aligned according to the same optical axis. Advantageously, no optical filter is placed in front of the second detector 42.

Some of the elements that are found on the optical path carry out an enlargement of the object at the image. The object is the light source 40 and the image is the photosensitive face, respectively of the first detector 41 and of the second detector 42. The components that participate in this enlargement are substantially the lenses 400, 410, 420, diaphragms 411, 421 and the concave mirror 51.

The light source 40 is for example formed from 2 different LEDs, but complementary as to their spectral bands. These LEDs are doubled in order to form a single-block component of 4 LEDs of a higher power. The identical LEDs are disposed on the same diagonal in order to maximize the light power available over the entire frequency bandwidth when the axis of this beam is not perfectly centered on the various optical components in emission or in reception.

On the “measurement” optical path (the one that passes through the gas; beam portions F, F₁₁, F₁₂, F₁₃), the enlargement is carried out by the lenses 400 and 410, by the concave mirror 51 and by the first diaphragm 411. The image of the light source 40 is reproduced, in a diffused and deformed manner, after the image focal spot of the first lens 410, at the focusing distance of the measurement optical path F, F₁₁, F₁₂, F₁₃, exactly where the photosensitive face of the first detector 41 is placed.

On the “reference” optical path (the one that does not pass through the gas; beam portion F, F₂), the set of two lenses 400, 420 would provide a true image, not diffuse and not deformed, in particular in dimensions, of the 4 LEDs at a focusing distance from the reference optical path F, F₂, before the focal point of the receiver lens. As the light source 40 is 300 microns in diameter, and the sensitive face of the detector 42 is only 100 microns, this image would show to said detector only the center—black—of these 4 LED. So the detector 42 is moved away by a few millimeters in order to place it on the focal point of the lens 420 to diffuse the beam and to have a more homogeneous distribution of the light flow emitted by the 4 LEDs over the entire photosensitive face of the detector 42. This diffusion is done to the detriment of sharpness, but it is made possible by the excess light power of this very short path.

Thus, the distance between diaphragm and detector, can be longer for beam F₂ than for beam F₁₃. For this reason, the second diaphragm 421 at all points identical to the first diaphragm 411, differs however from the latter by the dimensions of the aperture thereof, depending on this distance. The second lens 420 is identical to the first lens 410.

On the “reference beam” side, due to the quasi collimation (rays that are quasi parallel between the separator 50 and the lens 420) made possible by the short optical path between the light source 40 and the detector 42, and carried out by the lens 400 on said light source, the distance between said lens 420 and the center of the separator cube 50 is of no importance. So this distance is greatly reduced, by nearly 1 mm for example, in order to contribute to the compactness of the chamber 10. On the other hand, on the “measurement beam” side, slightly converging, the distance between the lens 410 and the center of the separator cube 50 is linked to the distance between mirror and separator cube, and to the radius of curvature of the mirror (spherical).

The optical path through the gas has a total length of 40 cm, with a round trip in the gas zone measured, thanks to a mirror. This length is the result of a balance between the power of the LEDs, and a measurable absorption of the light, not too weak nor too strong, with respect to the density of the molecules located in the measured space, this for the range of pressures and temperatures respectively from 0 to 10 relative bar, and 3° C. to 50° C. No particular tolerance is imposed on this distance: deviations with respect to this distance are compensated during the calibration of the sensor.

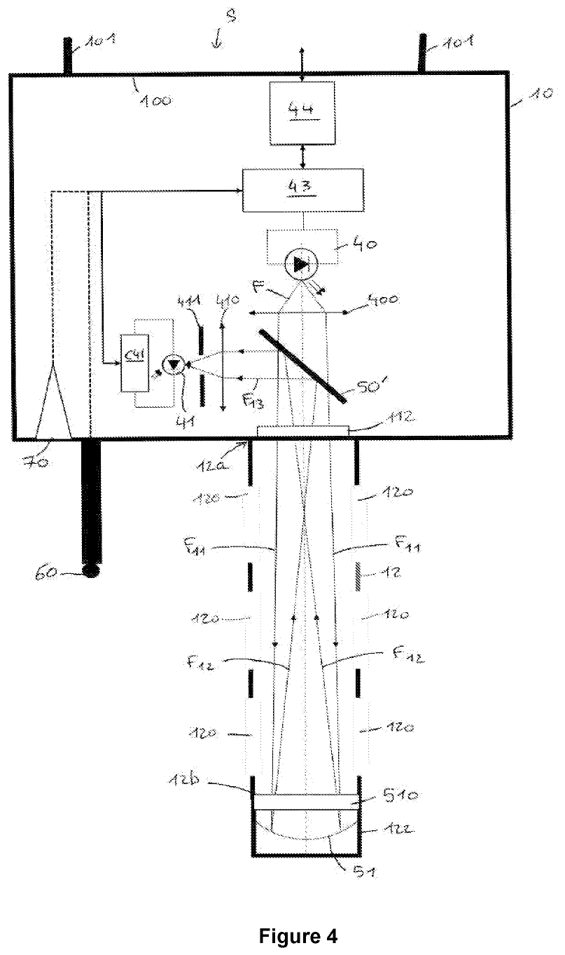

FIG. 4 shows an alternative embodiment of the probe S. Only the first detector 41 is used. In comparison with the embodiment of FIG. 3, there is no analysis of the light beam portion F₂. This solution is suitable when LEDs are used as a light source, when they are relatively stable, with little or no variation in their light intensity and/or frequency spectrum. The optical separator 50 is replaced with another optical element 50′ adapted to guide: towards the cage (12), the portion F₁₁ of the light beam F emitted by the light source 40; and towards the detector 41: the portion F₁₃ having propagated through the cage 12 and having interacted with the gas G. The optical element 50′ has for example the form of a semi-reflective sheet inclined 45° with respect to the incident beam F.

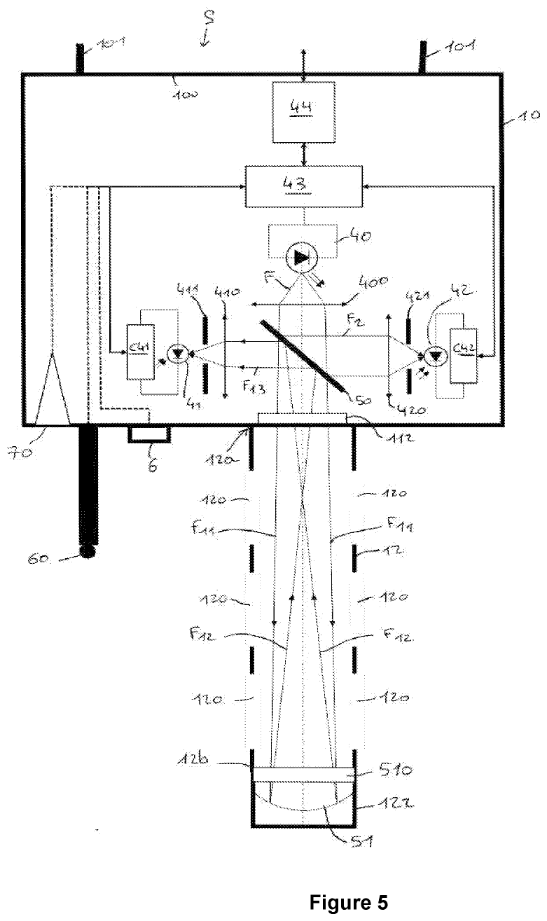

FIG. 5 shows yet another alternative embodiment of the probe S. A thermal conductivity sensor 6 is arranged on the probe S in such a way that it can interact with the gas G. This sensor 6 is more particularly fastened on the base 113 of the chamber 10, outside the cage 12, in such a way that it is not on the optical path. The sensor 6 is connected to the processing unit 43 in such a way that the signals that it emits are transmitted to said unit. The latter is adapted to generate measurement data of the composition of the gas G by taking account of these signals.

The probe of FIG. 5 is particularly adapted for measuring the amount of hydrogen present in the gas. Current regulations limit the amount of hydrogen (0% in England and in Belgium, 4% in Switzerland, 6% in France, 10% theoretically but 2% in practice in Germany) that can be injected into a gas distribution network. Actions are taken in the framework of energy transition, for example at the European level, in order to harmonize these limits and to increase them so as to make hydrogen useable on a large scale, as a massive means of storage of renewable energies. But, currently, gas apparatuses accept amount of hydrogen that are relatively low and generally do not support any sudden variations in this rate. The measurement of the amount of hydrogen in the channels wherein gas flows therefore appears relevant, and is made possible by the probe S.

As hydrogen does not have a molecular bond of atoms of different natures such as C—H, it cannot be detected by the spectrometer 4. As the thermal conductivity of hydrogen is exceptionally high with respect to that of methane, ethane, propane, butane, carbon monoxide, or carbon dioxide, the processing unit 43 is able to deduce the hydrogen content rather precisely, by knowing the content of the other compounds of the gas (by spectrometry analysis).

The thermal conductivity sensor 6, in its most effective realizations in the market, for example marketed under the reference XEN-TCG3880 from Xensor Integration Bv®, can offer a response time of less than one second. So, the probe S is capable of measuring instantaneous variations in the content in hydrogen in a variable mixture of oxidizing gas, and of allowing for the control of gas apparatuses (thanks to their actuators for controlling the flow rate of air and/or of gas, and/or ignition) in order to ensure the stability of the combustion. Like the spectrometer 4, the thermal conductivity sensor 6 does not require any particular maintenance or regular calibration in service, which makes it a component well suited for industrial application, with respect to other possibly more selective sensors, such as electro-chemical Hydrogen sensors, sensitive to saturation, to fowling and to poisoning described in aforementioned patent document U.S. Pat. No. 9,291,610 (ZELEPOUGA).

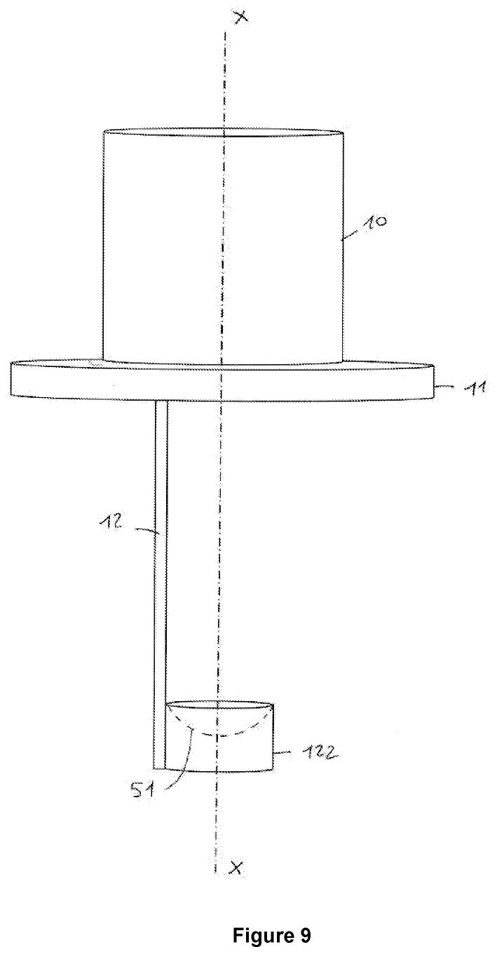

FIG. 9 shows an additional alternative embodiment of the probe S. The cage 12 is here simply formed from a rigid riser, preferably made from stainless steel, at the end of which is engaged the endpiece 122 wherein the optical reflector 51 is housed. This riser has for example the form of a rod or of a flat bar. The engagement of the endpiece on the riser can be carried out via welding, screwing, riveting, etc. The other end of the riser is engaged in the same way on the base of the chamber 10, in such a way that the optical reflector is aligned in the axis X-X. The riser is offset from this axis in order to not interfere with the optical path. This embodiment has the advantage of suppressing any obstacle (other than the riser) on the trajectory of the gas. However, this structure is less rigid and less robust than those shown in FIGS. 1a, 1b and 2 and has to be reserved for a vibration-free environment. It can also be provided to use two or more other risers to further rigidify the structure.

FIGS. 6A, 6B, 6C and 6D show the installation of the probe S on a channel C wherein the gas to be analyzed flows. The gas G and its direction of flow in the channel C are diagramed by the double arrow. The channel C can be that of a gas pipeline or, the gas supply channel of an apparatus A that uses a gas as fuel.

An aperture O is arranged on a wall of the channel C. This aperture O typically consists of a flange, or other arrangement initially provided for the installation of temperature or pressure probes for example. It can however be an aperture specifically designed for the probe S. The probe S is inserted into the aperture O in such a way that the cage 12 penetrates into the channel.

For a channel C of large diameter, for example greater than or equal to 200 mm, an installation according to the installation of FIG. 6A or 6D is favored. So that the cage 12 is passed through optimally by the gas G, the probe S is installed in such a way that the longitudinal axis X-X (which is also the axis of the optical path) of the elongate tube forming the cage 12 is perpendicular or substantially perpendicular (+/−10°) to the direction of flow of the gas G in the channel C. This configuration makes it possible to obtain particularly precise measurements.

For channels C of small diameter, for example less than 200 mm, an installation according to the embodiments of FIGS. 6B and 6C is favored. The channel C here has an elbow. The probe S is installed in such a way that the longitudinal axis X-X of the tube 12 is parallel or substantially parallel (+/−10°) to the direction of flow of the gas G in the elbow. Thanks to the turbulences created by the elbow of the channel C, this configuration makes it possible to obtained precise measurements. The choice of an installation according to FIG. 6B or according to FIG. 6C can depend on the space available around the channel C.

In the configuration of FIGS. 6A, 6B and 6C, the aperture O maintains the flange 11 at a distance from the inside if the channel C, in such a way that a portion of the cage 12 (portion located in the vicinity of the flange 11) is not in direct contact with the gas flow. This dead zone where the gas does not flow can, in certain cases, lengthen the response times. FIG. 6D shows a configuration that makes it possible to overcome these disadvantages. The aperture O and/or the flange 11 are shaped in such a way that said flange is flush with the internal wall of the channel C. The entire cage 12 is then in direct contact with the flow of gas in such a way that there are no longer any dead zones. Of course, the installation according to FIG. 6D also applies to the configurations of FIGS. 6B and 6C.

Once positioned at the aperture O, the probe S is maintained in position on the channel C. This maintaining in position is for example carried out by screwing or bolting the fastening flange 11 on an additional fastening flange arranged around the aperture O. A seal for the gas G flowing in the channel C, at the aperture O, is provided. This seal is in particular carried out by a seal J disposed between the two fastening flanges.

The measurement data generated by the probe S is advantageously transmitted to an electronic calculator UC of the apparatus A to which the channel C is connected. This electronic calculator UC is of a known type and in particular makes it possible to adjust, according to the type of apparatus, the ejection speed of the gas and/or of the air, the air-gas ratio, ignition advance, etc. The calculator UC will thus be able to instantly modify the settings and/or the operation of the apparatus A according to the data transmitted by the probe S.

The probe S described in the preceding paragraphs, with the technical solutions retained, is particularly inexpensive with respect to other similar probes known in the prior art. As its response time is less than one second, it is perfectly adapted for instantaneous measurements, directly in the flow of gas to be analyzed, without any sampling.

The measurements thus taken by spectrometry, and where applicable by thermal conductivity, are generally less precise than measurements taken by chromatography. Currently, gas chromatography is the most widely or commonly used method for the analysis and precise measurement of the composition of the combustible gases. The gas chromatography, however, typically requires at least several minutes to analyze a sample of gas and, consequently, does not provide information in real time. Combining the two techniques appears to be particularly effective as the applicant has been able to observe.

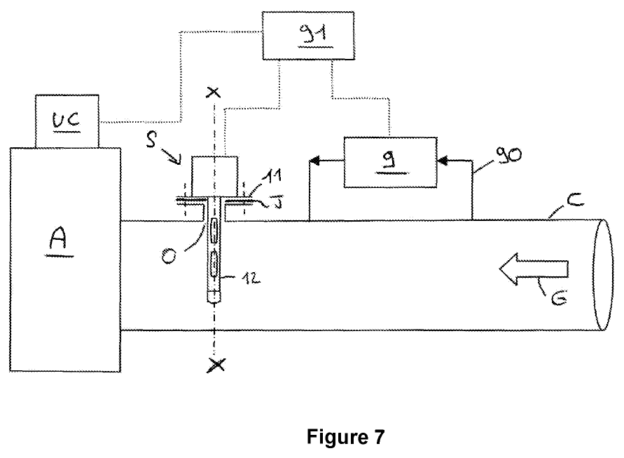

FIG. 7 shows an alternative embodiment where a gas chromatography apparatus 9 is connected to the channel C. This apparatus 9 is of the type known to those skilled in the art. A sample of gas is taken in the channel C using an injection system 90, then introduced into the detection system of the chromatograph 9. The latter will then generate precise measurement data of the composition of the gas.

The probe S and the chromatograph 9 are connected to a data processing unit 91. This processing unit 91 is similar to the processing unit 43 described hereinabove. It receives the measurement data generated by the probe S and the measurement data generated by the chromatograph 9. The measurement data generated by the probe S are received practically continuously (time interval less than 1 second) and the measurement data generated by the chromatograph 9 is received at a higher time interval (for example every 30 minutes). The processing unit 91 will then correct the measurement data generated by the probe S according to the measurement data generated by the chromatograph 9. This corrected data can then be communicated to the calculator UC of the apparatus A.

Graph 8 shows this correction. The solid line curve shows the continuous measurement of the concentration of the gas by the probe S, i.e. by spectrometry, and where applicable by thermal conductivity. It is observed that the concentration (C %) varies with the time (t). The points show one-off measurements (at instant t1, t2, t3 and t4) of the concentration of the gas by the chromatograph 9. The dotted line curves show the corrections made on the continuous measurement by spectrometry.

At instant t1, the processing unit 91 observes that the concentration measured by spectrometry is less than the concentration measured by chromatography. It will therefore adjust the results of the measurements by spectrometry, by correcting it with the delta (or difference) between the two measured concentrations. This delta will here be added to the results of the measurement by spectrometry.

At instant t2, the processing unit 91 observes that the concentration measured by spectrometry (and corrected at instant t1) is greater than the concentration measured by chromatography. The delta between the two measured concentrations will here be subtracted from the results initially corrected of the measurement by spectrometry.

At instant t3, the processing unit 91 observes that the concentration measured by spectrometry (and corrected at instant t2) correspond to the concentration measured by chromatography. The processing unit 91 therefore does not carry out any additional correction.

At instant t4, the processing unit 91 observes that the concentration measured by spectrometry (and corrected at instant t2) is again less than the concentration measured by chromatography. It will then readjust the results of the measurement by spectrometry, by adding to them the delta between the two measured concentrations.

Measurements are therefore obtained that are more precise than those obtained only by spectrometry and faster than those obtained only by chromatography. There is therefore a synergy of the two techniques in order to obtain in the end a continuous and very precise measurement of the concentration of the gas to be analyzed.

The same correction can be made on the measurement data of the amount of hydrogen deduced from the measurement data from the thermal conductivity sensor 6 and measurement data from the spectrometer 4. Indeed, the chromatography apparatus 9 is adapted to directly measure the amount of hydrogen of the gas. The processing unit 91 can then correct the measurement data of the amount of the hydrogen generated by the probe S according to the measurement data generated by the chromatograph 9. This correction is identical to that described hereinabove in reference to graph 8.

The arrangement of the various elements and/or means and/or steps of the invention, in the embodiments described hereinabove, must not be understood as requiring such an arrangement in all the implementations. In any case, it is understood that various modifications can be made to these elements and/or means and/or steps, without moving away from the spirit and scope of the invention. In particular:
The chamber 10 and/or the base 113 and/or the flange 11 and/or the tube forming the cage 12, are not necessarily of circular section. They can be of square, rectangular, oval, polygonal, etc. section.
The tube 12 can be directly engaged with the flange 11 in the case where the chamber 10 is devoid of base 113.
the probe S can be provided with another means of fastening on the channel rather than the flange 11. It is possible for example to arrange a threading on the external sidewall of the chamber 10, said threading is engaged into a complementary threading arranged in a tapping made in the wall of the channel
The cage 12 can have the form of a grated plate shaped in the form of a tube and/or holder of the optical reflector.
The aperture 121 can open directly into the chamber 10.
The cage 12 can be shaped in such a way that its second end 12b is initially closed off, without the adding of the endpiece 122, said second closed off end holds the optical reflector 51.
For a pressure P of the gas to be analyzed greater than 60 bars, the modification in the refractive index of the gas can lead to using means of lighting that are more powerful than LEDs. The light source 40 can in this case consist of a laser for example if higher costs are accepted. It is also possible to use a tungsten lamp or a halogen lamp for a vibration-free environment if shorter service life is accepted.
The guiding of all or a portion of the light beam F towards the detector or detectors 41, 42 of the spectrometer 4 and towards the cage 12, can be carried out by an arrangement of reflective sheets, or by an optical separator other than a separator cube (e.g.: separation sheet or plate).
At the risk of increasing the costs through complexity of the assembly of the probe S, it can be considered that the optical fibers be the interface between the light source 40 and the optical separator 50; and/or between the optical separator 50 and the cage 12; and/or between the optical separator 50 and the first detector 41; and/or between the optical separator 50 and the second detector 42.
The processing unit 43 can be offset from the chamber 10. In which case, the signals emitted by the detectors 41, 42 are transmitted to the processing unit 43 by wires or wirelessly (for example via Bluetooth, ISM, Wi-Fi, ANT, ZIGBEE, etc.). The measurement data of the composition of the gas is then generated outside the chamber 10.
The processing unit 43 and the data processing unit 91 can be the same processing unit.

According to embodiments not covered by the present invention, the arrangements and/or the characteristics of the various components installed in the chamber 10 and which have been described hereinabove, also apply to probes for measuring the composition of a gas that do not have a cage 12. In particular, the use of LEDs, the pulse emission of the light beam F, the particular arrangement of the motherboard, the use of shielded connection cables, filling the chambre 10 with a resin, the interfaces carried out by an air gap (between the light source 40 and the optical separator 50; and/or between the optical separator 50 and gas to be analyzed; and/or between the optical separator 50 and the first detector 41; and/or between the optical separator 50 and the second detector 42) with lenses and diaphragms, can perfectly be used in other types of probes, including those described in patent documents US2006/0092423 (SERVAITES), U.S. Pat. No. 8,139,222 (SAVELIEV) and U.S. Pat. No. 9,291,610 (ZELEPOUGA), or EP2198277 (SP3H).

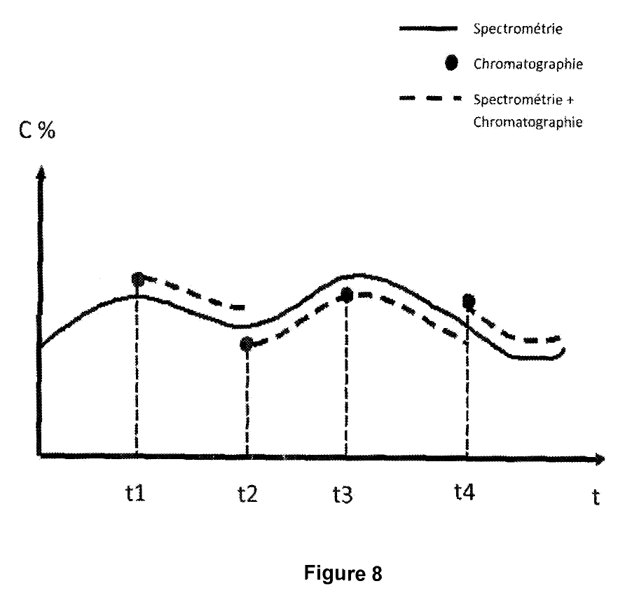

According to other embodiments not covered by the present invention, the assembly of FIG. 7 and the correction of the instantaneous measurements of the spectrometer with the one-off measurements of the chromatograph of FIG. 8, can be applied to other types of probes, including those described in patent documents US2006/0092423 (SERVAITES), U.S. Pat. No. 8,139,222 (SAVELIEV) and U.S. Pat. No. 9,291,610 (ZELEPOUGA), or EP2198277 (SP3H).

1.-19. (canceled)

20. A probe comprising a spectrometer adapted to measure the composition of an oxidizing gas after analysis of at least one portion of a light beam having interacted with said gas, said spectrometer comprising:
a light source adapted to emit a light beam,
an optical device including optical elements configured to:
guide, towards the gas to be analyzed, at least one portion of the light beam, and
guide, towards a detector, a portion of the light beam having interacted with the gas,
wherein:
the probe includes a cage adapted to be removably installed inside a channel wherein the gas to be analyzed flows,
the cage is configured to permit a flow of the gas through said cage,
at least one portion of the light beam propagates through the cage so as to be able to interact with the gas,
the cage serves as a holder for an optical reflector adapted to reflect, towards the detector, the portion of the light beam propagating through said cage and having interacted with the gas, and,
the cage is formed by an elongate tube the sidewall of which contains apertures distributed over the perimeter of said wall, said apertures are configured to allow the gas to pass radially through said tube, the combined area of said apertures representing more than 50% of the area of the wall of said tube.

21. The probe according to claim 20, wherein:
the optical device includes an optical element adapted to guide, towards a second detector of the spectrometer, a portion of the light beam emitted by the light source in such a way that said spectrometer also analyzes a light beam portion that does not propagate through the cage,
the spectrometer is adapted to measure the composition of the gas according to:
data resulting from the analysis of the light beam portion having propagated through the cage, and
data resulting from the analysis of the light beam portion (F₂) that does not propagate through the cage.

22. The probe according to claim 21, wherein the optical element is an optical separator placed between the light source and the cage, said optical separator is adapted to:
transmit in the cage, a portion of the light beam emitted by the light source,
deviate towards a first detector, the light beam portion having propagated through the cage and which is reflected by the optical reflector arranged in said cage, and
deviate towards the second detector of the spectrometer, the light beam portion that does not propagate through the cage.

23. The probe according to claim 20, wherein the optical device includes an optical element adapted to guide:
towards the cage: a portion of the light beam emitted by the light source, and
towards the detector: the light beam portion having propagated through the cage and which is reflected by the optical reflector arranged in said cage.

24. The probe according to claim 22, wherein:
an air gap serves as an interface between the light source and the optical separator, and
an air gap serves as an interface between the optical separator and the cage.

25. The probe according to claim 22, wherein:
an air gap serves as an interface between the optical separator and the first detector,
a first lens and a first diaphragm are disposed in the air gap that separates the optical separator and the first detector, said first lens and said first diaphragm being arranged in such a way that said beam portion having propagated through the cage and which is reflected by the optical reflector, first passes through said first lens then said first diaphragm before impacting said first detector.

26. The probe according to according to claim 22, wherein:
an air gap serves as an interface between the optical separator and the second detector,
a second lens and a second diaphragm are disposed in the air gap that separates the optical separator and the second detector, said second lens and said second diaphragm being arranged in such a way that the light beam portion that does not propagate through the cage, first passes through said second lens then said second diaphragm before impacting said second detector.

27. The probe according to claim 20, wherein:
a thermal conductivity sensor is arranged on the probe in such a way that said sensor can interact with the gas to be analyzed,
a data processing unit is adapted to generate measurement data of the composition of the gas by taking account of the signals emitted by the thermal conductivity sensor and the measurement data generated by the spectrometer.

28. The probe according to claim 20, further comprising:
a chamber sealed from the gas to be analyzed and wherein the spectrometer is installed, and
the cage includes a first end and a second end that are opposite, said first end includes an aperture that communique with the chamber, and
the optical reflector, the aperture and the light source are aligned along the same axis.

29. The probe according to claim 28, wherein:
a transparent window made from polished sapphire is disposed facing the aperture arranged at the first end of the cage, said window forms the gas seal between the chamber and said cage,
the portion of the light beam propagating through the cage, passes through the window.

30. The probe according to claim 20, wherein the optical reflector is installed on an endpiece, said endpiece is added onto an end of the cage.

31. The probe according to claim 20, wherein a transparent window made of polished sapphire covers the optical reflector.

32. The probe according to claim 20, including a chamber that is sealed from the gas to be analyzed and wherein the spectrometer installed, with more than 50% of the volume of said chamber being filled with resin or elastomer.

33. A system comprising a probe and a channel wherein a gas to be analyzed flows, wherein the probe is in accordance claim 20, the cage of said probe being removably installed inside the channel.

34. The system according to claim 33, wherein the cage has a longitudinal axis perpendicular or substantially perpendicular to the direction of flow of the gas in the channel.

35. The system according to claim 33, wherein:
the channel has an elbow,
the cage has a longitudinal axis parallel or substantially parallel to the direction of flow of the gas in the elbow of the channel.

36. The system according to claim 33, further comprising a gas chromatography apparatus adapted to generate measurement data of the composition of the gas, said apparatus is connected to the channel, in such a way that a sample of the gas flowing in said channel is analyzed by said apparatus.

37. The system according to claim 36, wherein:
the probe and the gas chromatography apparatus are connected to a data processing unit,
the processing unit is adapted to correct the measurement data generated by the probe according to the measurement data generated by the gas chromatography apparatus.

38. The system according to claim 36, wherein:
the probe further comprises:
a thermal conductivity sensor arranged in such a way that said sensor can interact with the gas to be analyzed, and
a data processing unit adapted to generate measurement data of the composition of the gas by taking account of the signals emitted by the thermal conductivity sensor and the measurement data generated by the spectrometer,
the probe and the gas chromatography apparatus are connected to a data processing unit, and the processing unit is adapted to correct said measurement data of the composition of the gas according to said measurement data generated by the gas chromatography apparatus.
